(19) United States
(12) Patent Application Publication
(10) Pub. No.: US 2025/0169889 A1
(43) Pub. Date: May 29, 2025

(54) METHOD, COMPUTING DEVICE, SYSTEM, AND COMPUTER PROGRAM PRODUCT FOR ASSISTING POSITIONING OF A TOOL WITH RESPECT TO A SPECIFIC BODY PART OF A PATIENT

(71) Applicant: UNIVERSITÄT ZÜRICH, PROREKTORAT FORSCHUNG, CH-8006 ZÜRICH (CH)

(72) Inventors: Hooman ESFANDIARI, Vancouver (CA); Philipp FÜRNSTAHL, Eglisau (CH); Mazda FARSHAD, Zumikon (CH)

(73) Assignee: UNIVERSITÄT ZÜRICH, PROREKTORAT FORSCHUNG, CH-8006 ZÜRICH (CH)

(21) Appl. No.: 18/808,218

(22) PCT Filed: Feb. 17, 2023
(86) PCT No.: PCT/EP2023/054056

§ 371 Date: Aug. 19, 2024

(30) Foreign Application Priority Data
Feb. 21, 2022 (CH) CH000171/2022

(51) Int. Cl. A61B 34/20; A61B 34/00; A61B 34/10; G06T 7/73; G06V 10/25; G06V 10/26; G06V 10/82

(52) U.S. Cl. CPC A61B 34/20; A61B 34/25; G06T 7/75; G06V 10/25; G06V 10/26; G06V 10/82; A61B 2034/102; A61B 2034/2065; G06T 2200/04; G06T 2207/10068; G06T 2207/10081; G06T 2207/10116; G06T 2207/10132; G06T 2207/20081; G06T 2207/20084; G06V 2201/03

(57) ABSTRACT

A computer-implemented method, computing device, system and computer program product for assisting positioning of a tool (5) with respect to a specific body part (202) of a patient (200) comprising: receiving intraoperative imaging data (ID) comprising 2D images capturing the specific body part (202) from a plurality of perspectives and 2D image(s) capturing at least a part of the tool (5) from at least one perspective; reconstructing an anatomical 3D shape (AS) of the specific body part (202) using an artificial-intelligence based algorithm corresponding to the specific body part (202) based on the intraoperative imaging data (ID) and data indicative of perspectives of 2D images; estimating a current position (5c) of the tool (5) with respect to the anatomical 3D shape (AS) based on the intraoperative imaging data (ID); and generating positioning guidance data (GD) comprising the estimated current position (5c) of the

### BACKGROUND OF THE INVENTION

### Field of the Invention

The present invention relates to a computer-implemented method of assisting positioning of a tool, such as a surgical tool, with respect to a specific body part of a patient. The pre-sent invention further relates to a computing device configured to assisting positioning of a tool, such as a surgical tool, with respect to a specific body part of a patient. The present invention even further relates to a system for assisting positioning of a tool with respect to a specific body part of a patient. The present invention even further relates a computer program product, comprising instructions, which, when carried out by a processing unit of a computing device, cause the computing device to assisting positioning of a tool, such as a surgical tool, with respect to a specific body part of a patient.

### Discussion of Related Art

Conventional, prior art computer assisted surgery and surgical navigation technology has enabled surgeons to have “glass-like” patients where important information from preoperative imaging and preoperative planning are directly available to the surgeon's perception. Conventional computer assisted surgical guidance systems rely on three major aspects: 1) preoperative planning based on anatomical 3D models derived from preoperative imagery (such as CT or MRI scans); 2) registration of preoperative data to intraoperative anatomy and 3) real-time tracking of surgical instruments.

A preoperative plan that is used in state-of-the-art surgical guidance systems, depicts the surgical process in a step-by-step fashion on generated 3D anatomical models. However, these preoperative plans are usually an idealized sketch of the intraoperative reality, which can be affected by pre-operative events affecting the respective body parts of a patient and/or intraoperative conditions such as: bleeding, complications and surgical inaccuracies. Therefore, surgeons are often forced to revert back to the conventional (non-navigated) techniques.

Registration of a preoperative plan to intraoperative anatomy is often considered as the other drawback of conventional computer assisted surgical guidance systems. In the context of the present application, the term registration refers to the process of calibration/alignment of a preoperative plan to the actual, real-life anatomy and physical location, orientation of the patient at the time of surgery. Conventional computer assisted surgical guidance systems address this through the use of techniques such as: matching mutual landmarks and/or features in the preoperative plan to 3D anatomical models using optically tracked pointers or through the use of image-based registration methods that automatically match the preoperative imagery (such as CT or MRI scans) to the intraoperatively acquired images (i.e. 2D-3D registration). However, such common techniques in computer assisted surgery and surgical guidance systems are susceptible to different sources of error or technical complications that range from marker movement and small capture range to slow computations.

Documents US 2022/044440 A1 and WO 2020/108806 A1 describe methods for artificial-intelligence assisted surgery using Statistical Shape Modeling, wherein an object identified (using an artificial-intelligence algorithm) from intraoperative imaging data is classified in an X-ray projection image, and by deforming a statistical shape model of the classified object to fit the imaging of the classified object in the X-ray image, a 3D representation as well as a localization of the classified object is determined. However, statistical shape modeling suffers from the disadvantage that individual features captured of an intraoperative image, that were are not captured by the statistical shape model, are lost in the process of deforming the statistical shape model to fit the intraoperative image. Hence, Statistical Shape Modeling can at best provide a general, statistical approximation of a 3D dimensional shape based on intraoperative imaging, but is not suitable for a 3D reconstruction of an anatomical shape.

In the third aspect of conventional computer assisted surgical navigation, tracking a three-dimensional pose of a tool, in particular the desired surgical hardware, should be estimated within the same reference frame of the anatomy of the specific body part of the patient. For this, known computer assisted surgery and surgical guidance systems rely on reference markers that are attached to the surgical tools. However, line-of-sight issues are noted as a significant burden for the clinical usage of such systems. Methods for surgical tool pose estimation based on 2D X-rays exist that use traditional (generally intensity-based) registration techniques. However, such techniques are prone to the same limitations as the image-based registration methods.

Recently, with the introduction of fluoroscopy machines that are capable of intraoperative Cone-Beam Computed Tomography CBCT, it is possible to acquire a 3D volumetric image of the patient during the operation and use this data in combination with optical tracking systems to provide registration-free surgical navigation. In such methods, the patient is registered to the preoperative plan through monitoring of patient-mounted, optically tracked reference markers, which are visible both in the intraoperative Cone-Beam Computed Tomography and by the optical tracking system. Due to the acquisition of a full-range Cone-Beam Computed Tomography image, such methods result in added ionizing radiation and also can suffer from metal artifact issues. Moreover, a major technical limitation of Cone-Beam Computed Tomography-based intraoperative navigation is the assumption of fixed reference markers attached to the patient anatomy, which is proven insufficient in operating room conditions.

Although these methods have been proven to result in superior implantation accuracies compared to standard free-hand surgical methods, they have not been yet widely adopted in the state of the art operating rooms around the world. As reported in a world-wide survey, only 11% of the spine surgeries are performed using computer-assisted navigation systems and the majority of the operations are performed using the conventional free-hand open techniques in which the surgeons rely on their visual and tactile feedback to place the spinal implants into the pedicle regions. This is due to the fact that the aforementioned computer-assisted surgical navigation methods require either an extensive registration process to transfer the preoperative plan unto the anatomy and/or generally require external navigation hardware to be installed in the operating room. This can interfere with existing surgical workflows and can result in added operation time, radiation exposure and cost. For instance, the navigation systems that require the acquisition of Cone-Beam Computed Tomography scans during the surgery can lead to an increase in the surgical time by up to 8.2 minutes and result in 2.09 to 4.81 mSV of ionizing radiation.

In summary, known computer assisted surgery and surgical guidance systems are prone to line-of-sight and/or marker movement issues; require extensive registration of a preoperative plan; require external navigation hardware to be installed in the operating room and/or significantly interfere with the surgical workflow.

### SUMMARY OF THE INVENTION

It is an object of the present invention to provide a method, computing device, system, and computer program product for assisting positioning of a tool with respect to a specific body part of a patient which overcome one or more of the disadvantages of the prior art.

In particular, it is an object of the present invention to provide a registration-free method for assisting positioning of a tool with respect to a specific body part of a patient that can reconstruct an anatomical 3D shape and generate a visual representation of a position of the tool with respect to the specific body part(s) of a patient using only intraoperative imaging data, i.e. without the need for navigation hardware to be installed in the operating room.

According to the present disclosure, this object is addressed by the features of the independent claims. In addition, further advantageous embodiments follow from the dependent claims and the description.

In particular, this object is achieved by a computer-implemented method of assisting positioning of a tool, such as a surgical tool (e.g. a surgical drill, knife or surgical laser equipment) or a medical diagnosis tool with respect to a specific body part of a patient, the method comprising:
receiving intraoperative imaging data;
reconstructing an anatomical 3D shape using the intraoperative imaging data and using an artificial-intelligence based algorithm corresponding to the specific body part;
estimating a current position of the tool based on the intraoperative imaging data; and
generating positioning guidance data comprising a visual representation of the estimated current position of the tool with respect to the anatomical 3D shape of the specific body part(s).

In particular embodiments, the steps of receiving intraoperative imaging data; estimating a current position of the tool and generating guidance data are carried out repeatedly or continuously for a period of time in preparation of/preceding a surgical treatment of the patient.

### Receiving Intraoperative Imaging Data

Intraoperative imaging data is received by a computing device from an imaging device arranged in the proximity of the patient. The imaging device being arranged in the proximity of the patient hereby refers to a positioning allowing the imaging device to capture the intraoperative imaging data of the patient. The imaging data comprises a plurality of 2D images. Two or more of the 2D images capture the specific body part of the patient from two or more different perspectives with respect to the specific body part of the patient. One or more of the same plurality of 2D images capturing the specific body part also capture at least a part of the tool from at least one perspective. In the context of the present invention, the term perspective, with respect to a perspective of the imaging data, refers to a location (e.g. in an x, y, and z Cartesian coordinate system) and/or orientation (e.g. roll, pitch, yaw) of the imaging device relative to the specific body part, respectively relative to the tool.

According to embodiments of the present invention, the intraoperative imaging data comprises one or more of: a) radiation-based images, in particular X-ray image(s); b) ultrasound image(s); c) arthroscopic image(s); d) optical imagery; and/or e) any other cross-sectional imagery. Images of the specific body part of the patient respectively a part of the tool are captured using an imaging device communicatively connected to the computing device. In case of radiation-based images, the imaging device may comprise a C-arm imaging device based on X-ray technology. The C-arm imaging device comprises a generator (X-ray source) and an image intensifier or flat-panel detector. A C-shaped connecting element allows movement horizontally, vertically and/or around a swivel axes, so that 2D X-ray images of the patient can be produced from various perspectives around the patient. The generator emits X-rays that penetrate the patient's body. The image intensifier or detector converts the X-rays into a visible image that is transmitted to the computing device.

The intraoperative imaging data comprises data indicative of perspectives corresponding to the plurality of the 2D images, identifying the location and/or orientation of the imaging device that captures plurality of the 2D images, such as a location in an x, y, and z Cartesian coordinate system and/or orientation as roll, pitch, yaw of the imaging device relative to the specific body part. According to embodiments disclosed herein, the data indicative of the perspectives corresponding to the plurality of the 2D images is stored in a datastore comprised by or communicatively connected to the computing device. Alternatively, or additionally, the perspectives corresponding to the intraoperative imaging data are estimated by the computing device based on the intraoperative imaging data.

In a particular embodiment, estimating perspectives corresponding to the intraoperative imaging data is performed using a tool geometrical model indicative of a geometry of the tool. First, a plurality of projections of the tool geometrical model are computed from a plurality of candidate perspectives. Candidate perspectives are selected as discrete perspectives within a predefined space of possible perspectives of the imaging device. In other words, non-realistic locations and orientations of the imaging device relative to the patient are not considered to conserve computing power. Thereafter, the perspectives corresponding to the 2D images of the intraoperative imaging data are identified by comparing the at least part of the tool as captured by the respective 2D image of the intraoperative imaging data with the plurality of projections computed from the plurality of candidate perspectives. In particular, the comparison comprises applying a matching function to identify a best match between one of the computed projections of the “virtual” tool (based on the tool geometrical model) from the plurality of candidate perspectives and the part of the “physical” tool as captured by the imaging device. The candidate perspective producing the best match is selected as the estimated perspective.

According to embodiments disclosed herein, estimating perspectives corresponding to the intraoperative imaging data is performed using an artificial-intelligence based algorithm trained using a multitude of imaging data sets with known perspectives. In order to overcome the limitations of the availability and/or accuracy of imaging data sets with known perspectives, a multitude of imaging data sets comprising 2D images from known perspectives are generated from 3D imaging data, in particular computed tomography CT scans. Using this artificial-intelligence based algorithm, trained prior to the surgery, the intraoperative position of the imaging device can be estimated only based on the intraoperative images without requiring neither an external tracking device nor a calibration phantom.

### Generating an Anatomical 3D Shape

An anatomical 3D shape of the specific body part is reconstructed by the computing device using an artificial-intelligence based algorithm corresponding to the specific body part based on the intraoperative imaging data and data indicative of the perspectives corresponding to the plurality of the 2D images.

According to embodiments disclosed herein, the anatomical 3D shape is reconstructed as a voxelized volume and/or mesh). It is important to emphasize that the artificial-intelligence based algorithm must be a model corresponding to the specific body part, enabling a reconstruction of a 3D anatomical shape from a plurality of 2D images of the specific body part.

According to particular embodiments disclosed herein, the artificial-intelligence based algorithm is trained using a multitude of annotated imaging data sets capturing body parts (of persons other than the patient) corresponding to the specific body part of the patient. The annotations of the imaging data sets comprise data identifying and/or describing properties of the body part, such as identifying pixels, vectors, contours, surfaces within 2D images and/or voxels capturing the specific body part within 3D images capturing the specific body part.

In order to overcome the limitations of the availability and/or accuracy of annotated imaging data sets capturing body parts corresponding to the specific body part of the patient, according to embodiments disclosed herein, a multitude of annotated imaging data sets are generated from annotated 3D imaging data, in particular computed tomography CT scans, capturing body parts corresponding to the specific body part of the patient. In particular, synthetic 2D images, such as fluoroscopy shots (i.e., DRRs), are generated from different points of view around the patient given an input preoperative CT scan. For example, using this method, up to several hundred annotated “synthetic” 2D images (capturing the specific body part) can be generated from a single annotated CT scan, which are annotated “synthetic” 2D images that can be used by the artificial-intelligence based algorithm to improve its capabilities of reconstructing accurate anatomical 3D shapes from as few 2D intraoperative images as possible.

According to embodiments, reconstructing the anatomical 3D shape is performed in two stages: segmenting the intraoperative imaging data in order to identify the specific body part of the patient; and reconstructing the anatomical 3D shape further using the segmented intraoperative imaging data.

In order to segment the intraoperative imaging data, region(s) of interest are first identified within the intraoperative imaging data, the region(s) of interest containing the specific body part of the patient using an artificial-intelligence based detection and segmentation model, such as a convolutional neural network based detection and segmentation model. The region(s) of interest are then semantically segmented using the artificial-intelligence based detection and segmentation model to thereby generate the segmented intraoperative imaging data.

### Estimating a Current Position of the Tool

Having reconstructed the anatomical 3D shape, a current position of the tool with respect to the anatomical 3D shape of the specific body part is estimated based on the intraoperative imaging data, in particular 2D images of the imaging data capturing of the tool. According to embodiments disclosed herein, estimating the current position of the tool is performed using a tool geometrical model indicative of a geometry of the tool. First, a projection of the tool geometrical model is compared with the at least part of the tool as captured by the respective 2D image of the intraoperative imaging data. The tool geometrical model being projected onto the plane(s) of one or more of the 2D images of the intraoperative imaging data capturing the at least part of the tool. The plane of the 2D images of the intraoperative imaging data is determined based on the perspective of each 2D image. Thereafter, a position of the tool geometrical model that produces a projection onto the planes of the 2D images of the intraoperative imaging data is determined that (best) matches the at least part of the tool as captured by the respective 2D image of the intraoperative imaging data. In other words, the reverse process is applied as compared to the (initial) determination of the perspectives of the intraoperative imaging data. However, this reverse process is applied not necessarily on the same 2D images (of the intraoperative imaging data) as the 2D images used for determining the perspectives of the images used for reconstruction of the anatomical 3D shape.

According to embodiments disclosed herein, while the anatomical 3D shape is reconstructed once, in an initial stage of the method of assisting positioning of the tool, the estimation of the current position of the tool is carried out repeatedly at set intervals and/or triggered by certain events and/or manually triggered.

In order to improve the accuracy of estimating the position of the tool and/or to improve the accuracy of estimating perspectives corresponding to the intraoperative imaging data, according to further embodiments, the method of the present invention further comprises providing a tool in accordance with a tool geometrical model. Wherein the tool geometrical model is specifically designed to optimize the estimation of its position based on as few intraoperative images as possible. In particular, the tool is designed such that at least a part thereof is not completely rotationally symmetric around any of the axis of the Cartesian coordinate system in order to allow estimation of the tool's orientation based on 2D images. Alternatively, or additionally, the tool is designed to comprise special markers to facilitate its identification based on 2D intraoperative images.

### Generating Positioning Guidance Data

Having reconstructed the anatomical 3D shape of the specific body part and having estimated the current position of the tool, positioning guidance data is reconstructed by the computing device, comprising a visual representation of the estimated current position of the tool with respect to the anatomical 3D shape of the specific body part(s). According to embodiments disclosed herein, the guidance data is reconstructed as a 2D image to be displayed on a computer display. Alternatively, or additionally, the guidance data is reconstructed as an augmented reality overlay, comprising overlay metadata allowing an augmented reality device, such as a headset, to project the overlay onto the field of view of a user such that the overlay is aligned with the user's view of the specific body part of the patient and/or aligned with the user's view of the tool. According to embodiments disclosed herein, the visual representation of the estimated current position of the tool is overlaid onto a visual representation of the reconstructed anatomical 3D shape.

According to embodiments disclosed herein, the computing device controls a display device to display at least part of the guidance data, the display device is a computer screen, an augmented reality headset, or any device configured to display guidance data.

In order to guide surgeons to correctly position the tool, according to further embodiments disclosed herein, a prescribed position of the tool with respect to the anatomical 3D shape of the specific body part is identified by the computing device and a visual representation of the prescribed position of the tool is overlaid onto a visual representation of the estimated current position of the tool. The prescribed position of the tool is retrieved or received by the computing device from a datastore comprised by or communicatively connected to the computing device. Alternatively, or additionally, the prescribed position of the tool is computed by the computing device, the prescribed position of the tool being determined by an optimization function based on the anatomical 3D shape of the body part(s) as well as data indicative of a surgical procedure.

Embodiments disclosed herein are advantageous, as they enable automatically performing a pre-surgical planning based on reconstructed anatomical 3D shapes and guiding surgeons in placement of surgical tool(s). Neither a preoperative planning stage is required to define the safe implant trajectories nor is a registration of preoperative data to the intraoperative patient's position is needed, given that the intraoperative imaging data is used to reconstruct the anatomical 3D shape of the body part (e.g. spine), based on which prescribed positions/trajectories of the tool(s) can be identified.

It is a further object of the present invention to provide a computing device for positioning of a tool with respect to a specific body part of a patient that can reconstruct an anatomical 3D shape and generate a visual representation of a position of the tool with respect to the specific body part(s) of a patient using only intraoperative imaging data, i.e. without the need for navigation hardware to be installed in the operating room and without the need for a registration process of a preoperative plan.

According to the present disclosure, this object is addressed by the features of the independent claims. In addition, further advantageous embodiments follow from the dependent claims and the description.

In particular, the above-identified object is further achieved by a computing device comprising: a data input interface; a data output interface; a processing unit; and a storage unit. The data input interface, such as a wired (e.g. Ethernet, DVI, HDMI, VGA) and/or wireless data communication interface (e.g. 4G, 5G, Wifi, Bluetooth, UWB), is communicatively connectable with an imaging device and configured to receive intraoperative imaging data therefrom. The data output interface such as a wired (e.g. Ethernet, DVI, HDMI, VGA) and/or wireless data communication interface (e.g. 4G, 5G, Wifi, Bluetooth, UWB) is configured to transmit at least part of guidance data to a display device communicatively connectable to the data output interface. The storage unit comprising instructions, which, when carried out by the processing unit, causes the computing device to carry out the method of assisting positioning of a tool according to any one of the embodiments disclosed herein.

According to embodiments, the computing device is a stand-alone computer communicatively connected to the imaging device. Alternatively, or additionally, the computing device is a remote computer (e.g. a cloud-based computer) communicatively connected to the imaging device using a communication network, in particular at least partially using a mobile communication network. Alternatively, or additionally, the computing device is integrated into the imaging device or the display device.

It is a further object of the present invention to provide a system for positioning of a tool with respect to a specific body part of a patient that can reconstruct an anatomical 3D shape and generate a visual representation of a position of the tool with respect to the specific body part(s) of a patient using only intraoperative imaging data, i.e. without the need for navigation hardware to be installed in the operating room and without the need for a registration process of a preoperative plan.

According to the present disclosure, this object is addressed by the features of the independent claims. In addition, further advantageous embodiments follow from the dependent claims and the description.

In particular, the above-identified object is further achieved by a system comprising: a computing device according to any one of the embodiments disclosed herein; an imaging device; and a display device, the system being configured to carry out the method according to any one of the embodiments disclosed herein. The imaging device is communicatively connected to the computing device and arranged in the proximity of the patient allowing the imaging device to capture the intraoperative imaging data of the patient such that two or more of the 2D images capture the specific body part of the patient from two or more different perspectives with respect to the specific body part of the patient. One or more of the same plurality of 2D images capturing the specific body part also capture at least a part of the tool from at least one perspective. In case of radiation-based images as intraoperative images, the imaging device comprises a C-arm imaging device based on X-ray technology. The C-arm imaging device comprises a generator (X-ray source) and an image intensifier or flat-panel detector. A C-shaped connecting element allows movement horizontally, vertically and/or around a swivel axes, so that 2D X-ray images of the patient can be produced from various perspectives around the patient. The generator emits X-rays that penetrate the patient's body. The image intensifier or detector converts the X-rays into a visible image that is transmitted to the computing device. The display device is a computer screen, an augmented reality headset, or any device configured to display a guidance data.

It is a further object of the present invention to provide a computer program product for positioning of a tool with respect to a specific body part of a patient that can reconstruct an anatomical 3D shape and generate a visual representation of a position of the tool with respect to the specific body part(s) of a patient using only intraoperative imaging data, i.e. without the need for navigation hardware to be installed in the operating room and without the need for a registration process of a preoperative plan.

According to the present disclosure, this object is addressed by the features of the independent claims. In addition, further advantageous embodiments follow from the dependent claims and the description.

In particular, the above-identified object is addressed by a computer program product, comprising instructions, which, when carried out by a processing unit of a computing device, cause the computing device to carry out the method according to any one of the embodiments disclosed herein.

According to embodiments, the instructions (comprised by the computer program product) comprise an artificial-intelligence based algorithm corresponding to a specific body part of a patient, the artificial-intelligence based algorithm having been trained using a multitude of annotated imaging data sets capturing body parts corresponding to the specific body part of the patient, wherein the annotations comprise data identifying and/or describing properties of the body part.

According to embodiments, the instructions (comprised by the computer program product) comprise instructions to control an imaging device to capture intraoperative imaging data comprising 2D images, a plurality of the 2D images capturing the specific body part of the patient from a plurality of different perspectives with respect to the specific body part of the patient and one or more of the plurality of the 2D images capturing at least a part of the tool from at least one perspective.

According to embodiments, the instructions (comprised by the computer program product) comprise instructions to control a display device such as to display at least part of the guidance data comprising a visual representation of the estimated current position of the tool, a visual representation of the reconstructed anatomical 3D shape and/or a visual representation of the prescribed position of the tool.

It is to be understood that both the foregoing general description and the following detailed description present embodiments, and are intended to provide an overview or framework for understanding the nature and character of the disclosure. The accompanying drawings are included to provide a further understanding, and are incorporated into and constitute a part of this specification. The drawings illustrate various embodiments, and together with the description serve to explain the principles and operation of the concepts disclosed.

The term “particular” as used in the present specification refers to embodiments of the invention, without any indication of preference or indication that features introduced as particular would be essential to all embodiments of the invention.

### BRIEF DESCRIPTION OF SEVERAL VIEWS OF THE DRAWINGS

The herein described invention will be more fully understood from the detailed description given herein below and the accompanying drawings which should not be considered limiting to the invention described in the appended claims.

FIG. 1 shows a highly schematic perspective view of a system for assisting positioning of a tool as installed in an operating room, according to an embodiment of the pre-sent invention;

FIG. 2 shows a flowchart illustrating steps of a method of assisting positioning of a tool, according to an embodiment of the present invention;

FIG. 3 shows a flowchart illustrating steps of reconstructing an anatomical 3D shape based on the intraoperative imaging data and data indicative of the perspectives corresponding to the plurality of the 2D images, according to an embodiment of the present invention;

FIG. 4 shows a schematic illustration of segmenting intraoperative imaging data in order to identify the specific body part of the patient;

FIG. 5 shows a schematic illustration of a further embodiment of segmenting intraoperative imaging data, comprising identification of region(s) of interest followed by semantically segmenting the region(s) of interest in order to identify the specific body part of the patient within the region(s) of interest;

FIG. 6 shows a schematic illustration of the reconstruction of an anatomical 3D shape based on segmented intraoperative imaging data using an artificial-intelligence based algorithm corresponding to the specific body part;

FIG. 7 shows a flowchart illustrating steps of a further embodiment of reconstructing an anatomical 3D shape in multiple stages;

FIG. 8 shows a schematic illustration of determining a prescribed position of the tool;

FIG. 9A shows an illustrative example of a visual representation of the estimated current position of the tool and a visual representation of the prescribed position of the tool overlaid onto a visual representation of the reconstructed anatomical 3D shape;

FIG. 9B shows an illustrative example of a visual representation of the estimated current position of the tool overlaid onto a 2D image of the intraoperative imaging data ID; and

FIG. 9C shows an illustrative example of a visual representation of the estimated current position of the tool, a visual representation of the prescribed position of the tool and a visual representation of an ideal screw trajectory of a surgical implant onto a visual representation of the reconstructed anatomical 3D shape.

### DETAILED DESCRIPTION OF THE INVENTION

Reference will now be made in detail to certain embodiments, examples of which are illustrated in the accompanying drawings, in which some, but not all features are shown. Indeed, embodiments disclosed herein may be embodied in many different forms and should not be construed as limited to the embodiments set forth herein; rather, these embodiments are provided so that this disclosure will satisfy applicable legal requirements. Whenever possible, like reference numbers will be used to refer to like components or parts.

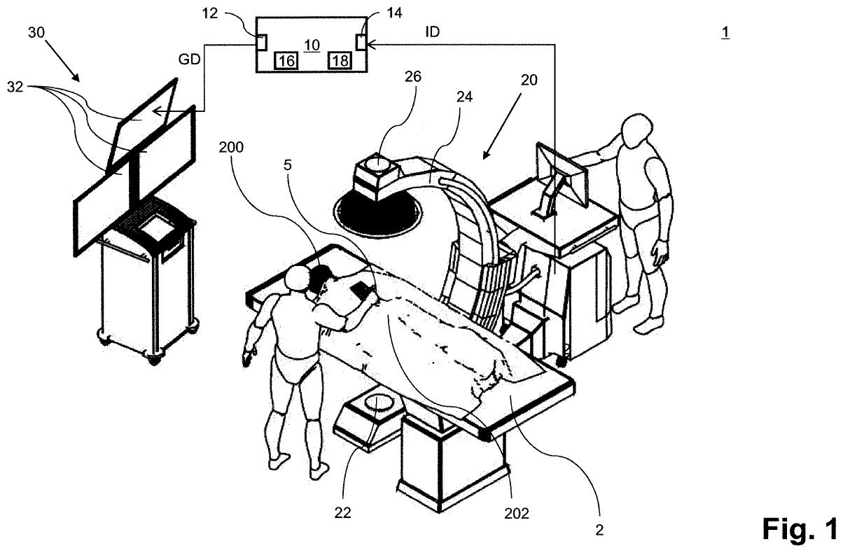

FIG. 1 shows a highly schematic perspective view of a system 1 for assisting positioning of a tool 5 as installed in an operating room, the patient 200 laying on an operating table 2. As illustrated, the system 1 comprises a computing device 10; an imaging device 20; and a display device 30. The system 1 is illustrated on the basis of an embodiment utilizing radiation-based images as intraoperative images. Accordingly, the imaging device 20 comprises a C-arm imaging device 20 capturing intraoperative images using X-ray technology. The C-arm imaging device 20 comprises a generator (X-ray source) 22. A C-shaped connecting element (C-arm) 24 allows movement horizontally, vertically and/or around a swivel axes, so that 2D X-ray images of the patient 200 can be produced from various perspectives around the patient. The generator 22 emits X-rays that penetrate the patient's body 200. A detector 26 converts the X-rays into imaging data ID that is transmitted to the computing device 10.

The imaging device 20 is communicatively connected to the computing device 10 and arranged in the proximity of the patient 200 allowing the imaging device 20 to capture the intraoperative imaging data ID of the patient 200 such that two or more of the 2D images capture the specific body part 202 of the patient 200 from two or more different perspectives with respect to the specific body part 202 of the patient 200. One or more of the same plurality of 2D images capturing the specific body part 202 also capture at least a part of the tool 5 from at least one perspective.

In the illustrated embodiment, the display device 30 comprises a series of computer screens 32 communicatively connected to the computing device 10 and configured to display guidance data GD.

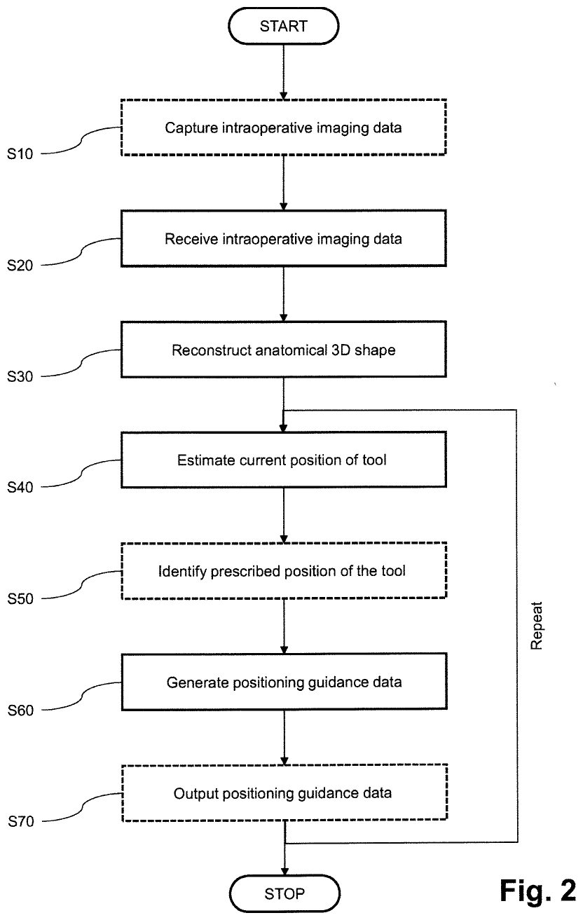

Turning now to the flowchart of FIG. 2, the steps of the computer-implemented method of assisting positioning of a tool 5 with respect to a specific body part 202 of a patient 200 shall be described.

As shown on FIG. 2, the method comprises the following major steps:
Step S10: capturing intraoperative imaging data;
Step S20: receiving intraoperative imaging data;
Step S30: reconstructing an anatomical 3D shape using the intraoperative imaging data and using an artificial-intelligence based algorithm corresponding to the specific body part 202;
Step S40: estimating a current position 5c of the tool based on the intraoperative imaging data ID;
Step S50: Identifying prescribed position 5p of the tool;
Step S60: generating positioning guidance data GD comprising a visual representation of the estimated current position 5c of the tool 5 with respect to the anatomical 3D shape AS of the specific body part 202; and
Step S70: outputting the positioning guidance data GD using a display device 30.

Steps specific to particular embodiments are illustrated on the figures with dashed lines.

In a step S10, intraoperative imaging data ID is captured by an imaging device 20 arranged in the proximity of the patient 200. The intraoperative imaging data ID comprises data indicative of perspectives corresponding to the plurality of the 2D images, identifying the location and/or orientation of the imaging device 20 that captures plurality of the 2D images, such as a location in an x, y, and z Cartesian coordinate system and/or orientation as roll, pitch, yaw of the imaging device 20 relative to the specific body part 202 of the patient 200.

According to a first embodiment, the data indicative of the perspectives corresponding to the plurality of the 2D images is stored in a datastore comprised by or communicatively connected to the computing device 10. The perspectives stored in a datastore are determined by tracking the C-arm 24 to estimate the imaging parameters at the time of exposure, through which the intraoperatively acquired 2D images can be assigned to their respective intrinsic and extrinsic imaging parameters that effectively define the perspectives from which the 2D images have been generated from. Optionally, the tracking the C-arm 24 is preceded by a calibration process, a preoperative calibration process (i.e., pre-calibration), in which the C-arm 24 is maneuvered in a specific manner to cover the indented range-of-motion. During this pre-calibration phase, the mathematical relationship between the tracking observations and the imaging parameters is established at specific pose intervals, which will be later used to derive an interpolation function that can produce the intraoperative imaging parameters based on the tracking data.

Alternatively, or additionally, the perspectives corresponding to the intraoperative imaging data ID are estimated by the computing device 10 based on the intraoperative imaging data ID. In an embodiment, a calibration algorithm extracts the perspectives of the 2D images by placing a precisely-fabricated calibration object (i.e., phantom), which include distinct features (e.g. radiopaque features) with known geometry, in the imaging field and estimate the imaging parameters based on the projection of those features.

Alternatively, or additionally, estimating perspectives corresponding to the intraoperative imaging data ID is performed using an artificial-intelligence based algorithm trained using a multitude of imaging data sets with known perspectives. In order to overcome the limitations of the availability and/or accuracy of imaging data sets with known perspectives, a multitude of imaging data sets comprising 2D images from known perspectives are generated from 3D imaging data, in particular computed tomography CT scans. For example, simulated intraoperative fluoroscopy shots (i.e. Digitally Reconstructed Radiographs, DRRs) generated based on preoperative CT scans along with their corresponding pose parameters are used to train Convolutional Neural Networks (CNNs) for regression tasks. Using this artificial-intelligence based algorithm, trained prior to the surgery, the intraoperative position of the imaging device 20 can be estimated only based on the intraoperative images without requiring neither an external tracking device nor a calibration phantom.

In a subsequent step S20, the intraoperative imaging data ID is received by the computing device 10 from the imaging device 20 via its data input interface 14.

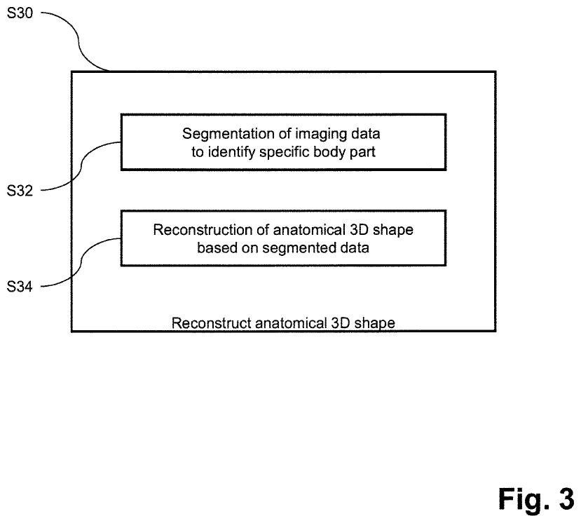

In subsequent step S30, an anatomical 3D shape AS of the specific body part 202 is reconstructed by the computing device 10 using an artificial-intelligence based algorithm corresponding to the specific body part 202 based on the intraoperative imaging data ID and data indicative of the perspectives corresponding to the plurality of the 2D images. A detailed description of step S30 of reconstructing an anatomical 3D shape AS is provided with reference to FIGS. 3, 4, 5, 6 and 7.

In step S40, the current position 5c of the tool 5 with respect to the anatomical 3D shape AS of the specific body part 202 is estimated based on 2D images of the imaging data ID capturing of the tool 5. The current position 5c of the tool 5 is performed based on prior knowledge of the geometry of the tool 5 as described by a tool geometrical model. First, a projection of the tool geometrical model is compared with at least a part of the tool 5 as captured by the respective 2D image of the intraoperative imaging data ID. The tool geometrical model being projected onto the plane(s) of one or more of the 2D images of the intraoperative imaging data ID capturing at least a part of the tool 5. The plane of the 2D images of the intraoperative imaging data ID is determined based on the perspective of each 2D image. Thereafter, a position of the tool geometrical model that produces a projection onto the planes of the 2D images of the intraoperative imaging data ID is determined that (best) matches the at least part of the tool 5 as captured by the respective 2D image of the intraoperative imaging data ID.

According to embodiments disclosed herein, while the anatomical 3D shape is reconstructed once, in an initial stage of the method of assisting positioning of the tool, the estimation of the current position 5c of the tool 5 is carried out repeatedly at set intervals and/or triggered by certain events and/or manually triggered.

In order to improve the accuracy of estimating the position of the tool 5, a tool 5 is proved in accordance with a tool geometrical model. Wherein the tool geometrical model is specifically designed to optimize the estimation of its position based on as few intraoperative 2D images as possible. In particular, the tool is designed such that at least a part thereof is not completely rotationally symmetric around any of the axis of the Cartesian coordinate system in order to allow estimation of the tool's orientation based on 2D images. Alternatively, or additionally, the tool is designed to comprise special markers to facilitate its identification based on 2D intraoperative images.

In step S50, a prescribed position 5p of the tool 5 with respect to the anatomical 3D shape AS of the specific body part 202 is identified by the computing device 10 and a visual representation of the prescribed position 5p of the tool 5 is overlaid onto a visual representation of the estimated current position 5c of the tool 5 in order to assist surgeons to correctly position the tool 5.

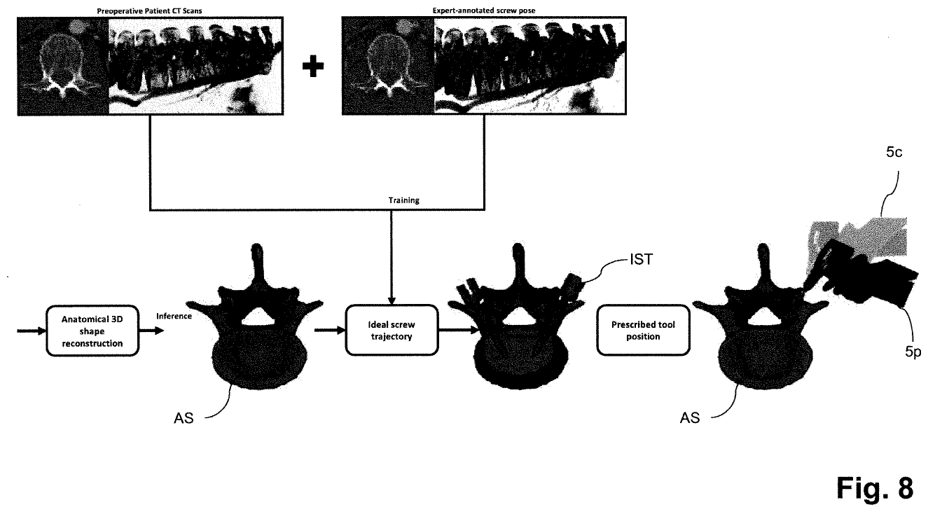

An embodiment of determining of the prescribed position 5p of the tool 5 is described with reference to FIG. 8.

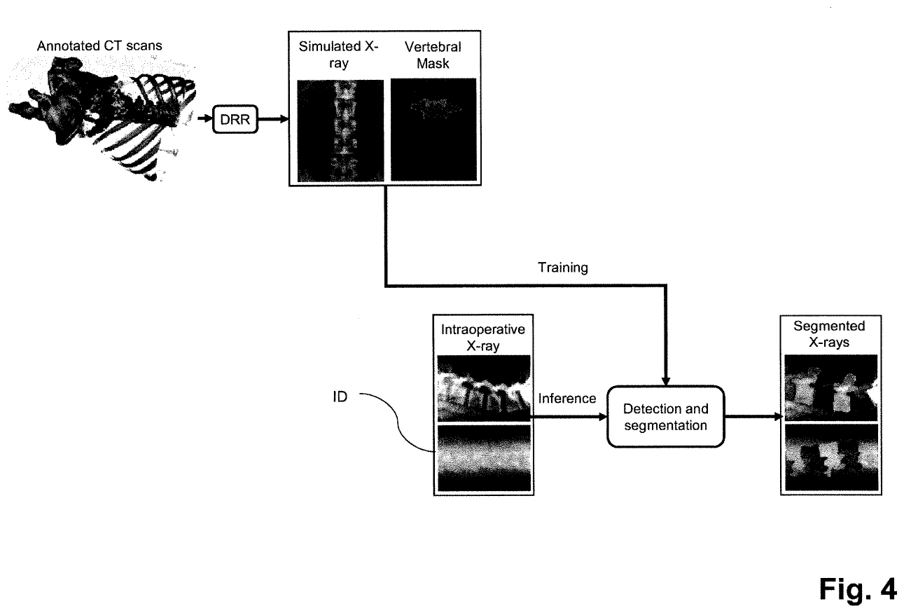

FIG. 3 shows a flowchart illustrating steps of reconstructing an anatomical 3D shape AS based on the intraoperative imaging data ID and data indicative of the perspectives corresponding to the plurality of the 2D images. As illustrated, reconstructing the anatomical 3D shape AS is performed in two stages: Step S32, segmenting the intraoperative imaging data ID in order to identify the specific body part 202 of the patient 200; and step S34, reconstructing the anatomical 3D shape AS further using the segmented intraoperative imaging data ID. Step S32, segmenting the intraoperative imaging data ID in order to identify the specific body part 202 of the patient 200 using an artificial-intelligence based detection and segmentation model is illustrated on FIG. 4, as applied for segmenting intraoperative 2D images of a spine to identify the individual vertebrae. To train the artificial-intelligence based detection and segmentation model, synthetic X-rays (i.e., DRRs) are generated from different points of view around the patient 200 given an input preoperative CT scan. The CT scans used for this purpose may be collected through a public dataset that includes CT scans along with the corresponding vertebral level annotations. For example, using this method, a training database of more than 40,000 annotated 2D images may be created from only 200 preoperative CT scans.

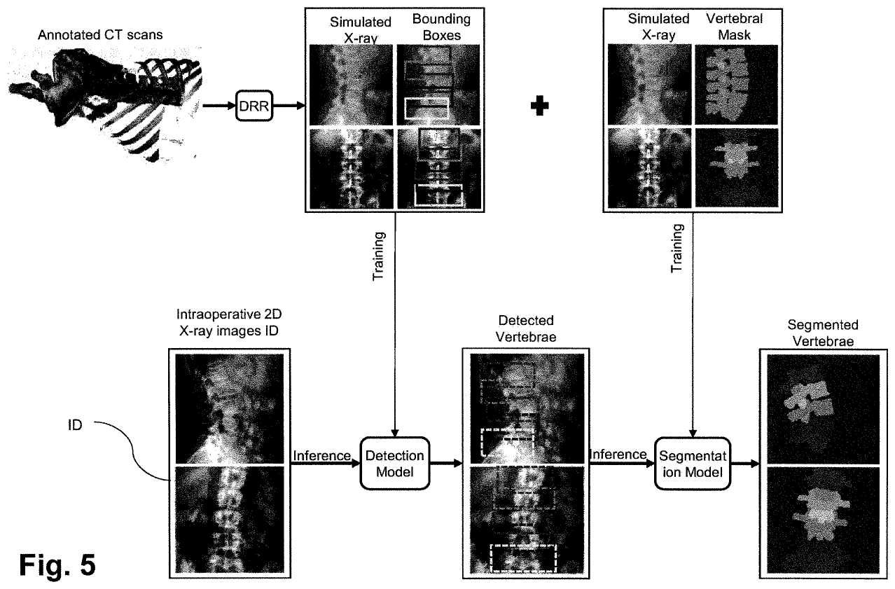

FIG. 5 shows a schematic illustration of a further embodiment of step S32 of segmenting intraoperative imaging data ID, according to a two-phase approach, comprising identification of region(s) of interest followed by semantically segmenting the region(s) of interest in order to identify the specific body part 202 of the patient 200 within the region(s) of interest. In order to segment the intraoperative imaging data ID, region(s) of interest are first identified within the intraoperative imaging data ID, the region(s) of interest containing the specific body part 202 of the patient 200 using an artificial-intelligence based detection and segmentation model, such as a convolutional neural network based detection and segmentation model. The region(s) of interest are then semantically segmented using the artificial-intelligence based detection and segmentation model to thereby generate the segmented intraoperative imaging data ID. Supervised learning is used to train the artificial-intelligence based detection and segmentation model used for the segmentation of step S32. First, a convolutional neural network CNN-based detection model is trained to identify the individual body parts (vertebral levels on the illustrated example) on the 2D images by detecting coordinates of bounding boxes each including a single body part (single vertebra). The identified bounding boxes are then used as a region of interest to crop the intraoperative 2D images. Furthermore, an end-to-end segmentation model is trained to semantically segment the projection of the vertebrae within the region of interest. During the inference phase, the intraoperative X-ray images are fed into the segmentation model, which produces semantic segmentations for each vertebral level (to be used for the 3D reconstruction purposes).

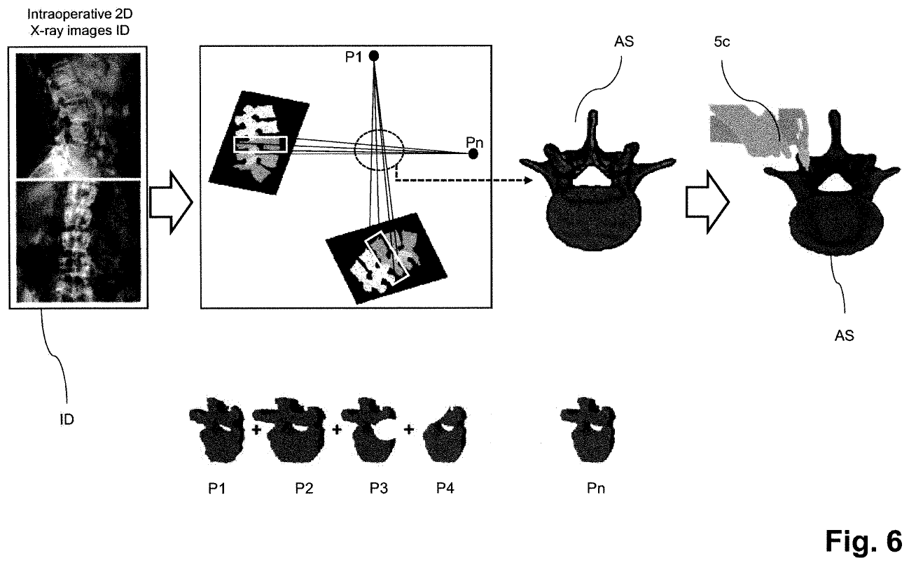

FIG. 6 shows a schematic illustration of the reconstruction of the anatomical 3D shape AS of the specific body part 202 using an artificial-intelligence based algorithm corresponding to the specific body part 202 based on the segmented intraoperative imaging data ID and data indicative of perspectives P₁₋ₙ corresponding to the plurality of the 2D images. As illustrated, the segmented intraoperative imaging data ID are back projected to a 3D coordinate system to create the anatomical 3D shape for each body part 202 (vertebrae). The back projection is performed on the basis of the perspective P₁₋ₙ of the 2D images, each 2D image providing information on the body part 202 from its perspective. Hence, the anatomical 3D shape AS is constructed incrementally, the more 2D images being comprised by the intraoperative data ID, the more precise the reconstructed anatomical 3D shape AS gets, as illustrated in the sequence of partial anatomical 3D shapes on the lower part of FIG. 6.

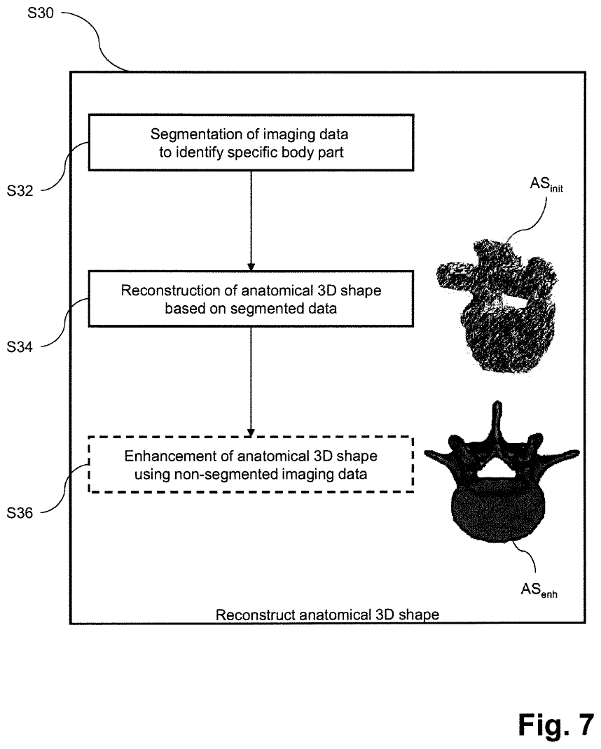

In addition to steps S32 and S34 as described with reference to FIG. 3, according to further embodiments as illustrated on the flowchart of FIG. 7, in a further step S36, an initial reconstruction of the anatomical 3D shape ASᵢₙᵢₜ is further enhanced using non-segmented imaging data. Given the potential errors in calibration and segmentation, a 3D shape enhancement model is employed in order to enhance the quality of the reconstructed initial anatomical 3D shape ASᵢₙᵢₜ. The 3D shape enhancement model, in particular a Convolutional Neural Network CNN architecture, is provided with two input streams. The first input stream comprises the initial reconstruction of the anatomical 3D shape ASᵢₙᵢₜ. The second input stream comprises the 2D segmentations of the specific body part 202 on the 2D images of the intraoperative imaging data ID. This way, the 3D shape enhancement model is trained to complete the missing components of the initial reconstruction (given the possibility of data loss in the initial reconstruction process due to missing projective views), by injecting patient specific shape information that are preserved in the original intraoperative 2D images to thereby reconstructe an enhanced anatomical 3D shape ASₑₙₕ.

Turning now to FIG. 8, an embodiment of determining of the prescribed position 5p of the tool 5 is described with reference to a use case of a surgical procedure of implanting a pedicle screw into a vertebra of a patient 200. The prescribed position 5p of the tool 5 is determined by an artificial-intelligence based optimization function based on the anatomical 3D shape AS of the specific body part 202 as well as data indicative of a surgical procedure.

Determining the prescribed position 5p of the tool 5 based on the anatomical 3D shape AS is advantageous as it has the potential to improve the surgical workflow by forsaking the need for a preoperative scan and the corresponding manual process, which can be both costly and time-consuming. Supervised learning and Reinforcement Learning RL is used to train the artificial-intelligence based optimization function based on a clinical dataset consisting of expert-identified ideal screw trajectories. The prescribed position 5p of the tool 5 is then determined based on the ideal screw trajectory IST further based on prior knowledge of the geometry of the tool 5.

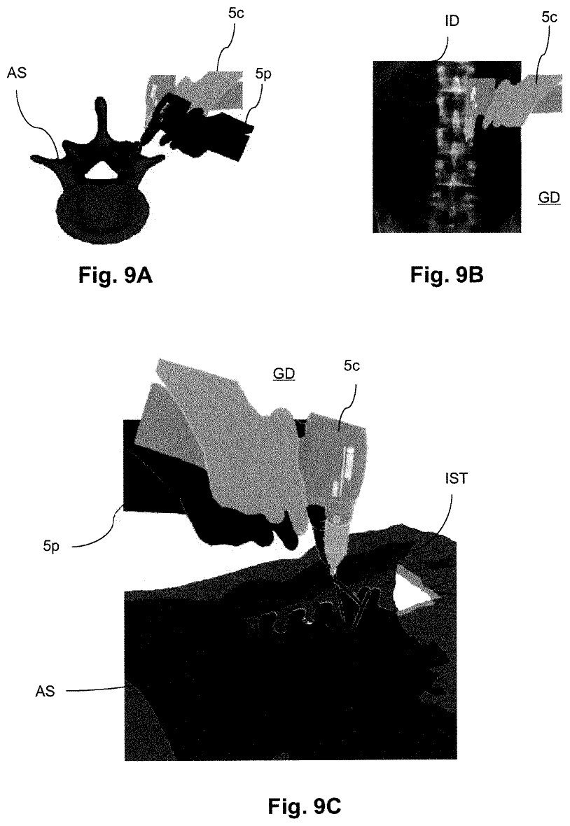

FIGS. 9A, 9B and 9C show embodiments of the positioning guidance data GD. FIG. 9A shows positioning guidance data GD comprising a visual representation of the estimated current position 5c of the tool 5 and a visual representation of the prescribed position 5p of the tool 5 overlaid onto a visual representation of the reconstructed anatomical 3D shape AS.

FIG. 9B shows positioning guidance data GD comprising a visual representation of the estimated current position 5c of the tool 5 overlaid onto a 2D image of the intraoperative imaging data ID.

FIG. 9C shows positioning guidance data GD comprising a visual representation of the estimated current position 5c of the tool 5, a visual representation of the prescribed position 5p of the tool 5 and a visual representation of an ideal screw trajectory of a surgical implant onto a visual representation of the reconstructed anatomical 3D shape AS.

1. A computer-implemented method of assisting positioning of a tool (5) with respect to a specific body part (202) of a patient (200), the method comprising:
a) receiving, by a computing device (10), intraoperative imaging data (ID) from an imaging device (20) arranged in a proximity of the patient (200), the intraoperative imaging data (ID) comprising 2D images, a plurality of the 2D images capturing the specific body part (202) of the patient (200) from a plurality of different perspectives with respect to the specific body part (202) of the patient (200) and one or more of the plurality of the 2D images capturing at least a part of the tool (5) from at least one perspective;
b) reconstructing, by the computing device (10), an anatomical 3D shape (AS) of the specific body part (202) using an artificial-intelligence based algorithm corresponding to the specific body part (202) based on the intraoperative imaging data (ID) and data indicative of perspectives corresponding to the plurality of the 2D images;
c) estimating, by the computing device (10), a current position (5c) of the tool (5) with respect to the anatomical 3D shape (AS) of the specific body part (202) based on the intraoperative imaging data (ID); and
d) generating, by the computing device (10), positioning guidance data (GD) comprising a visual representation of the estimated current position (5c) of the tool (5) with respect to the anatomical 3D shape (AS) of the specific body part (202).

2. The computer-implemented method of assisting positioning of a tool (5) with respect to a specific body part (202) of a patient (200) according to claim 1, wherein the step of reconstructing, by the computing device (10), an anatomical 3D shape (AS) of the specific body part (202) comprises training the artificial-intelligence based algorithm using a multitude of annotated imaging data sets capturing body parts corresponding to the specific body part (202) of the patient (200), wherein the annotations comprise data identifying and/or describing properties of the body part.

3. The computer-implemented method of assisting positioning of a tool (5) with respect to a specific body part (202) of a patient (200) according to claim 2, comprising generating a multitude of annotated imaging data sets—comprising 2D image(s) capturing body parts corresponding to the specific body part (202) of the patient (200) from a plurality of different perspectives—from annotated 3D imaging data, in particular computed tomography CT scans, capturing body parts corresponding to the specific body part (202) of the patient (200).

4. The computer-implemented method of assisting positioning of a tool (5) with respect to a specific body part (202) of a patient (200) according to claim 1, wherein the step of reconstructing the anatomical 3D shape (AS) comprises:
a) segmenting the intraoperative imaging data (ID) in order to identify the specific body part (202) of the patient (200); and
b) reconstructing the anatomical 3D shape (AS) further using the segmented intraoperative imaging data (ID).

5. The computer-implemented method of assisting positioning of a tool (5) with respect to a specific body part (202) of a patient (200) according to claim 4, wherein the step of segmenting the intraoperative imaging data (ID) comprises:
a) identifying region(s) of interest within the intraoperative imaging data (ID) containing the specific body part (202) of the patient (200) using at least one of an artificial-intelligence based detection and segmentation model, such as and a convolutional neural network based detection and segmentation model; and
b) semantically segmenting the region(s) of interest using the artificial-intelligence based detection and segmentation model to thereby generate the segmented intraoperative imaging data (ID).

6. The computer-implemented method of assisting positioning of a tool (5) with respect to a specific body part (202) of a patient (200) according to claim 1, further comprising estimating, by the computing device (10), the perspectives corresponding to the intraoperative imaging data (ID).

7. The computer-implemented method of assisting positioning of a tool (5) with respect to a specific body part (202) of a patient (200) according to claim 6, wherein estimating perspectives corresponding to the intraoperative imaging data (ID) is performed using a tool geometrical model indicative of a geometry of the tool (5) and comprises:
a) computing a plurality of projections of the tool geometrical model from a plurality of candidate perspectives; and
b) identifying the perspectives corresponding to the intraoperative imaging data (ID) by comparing the at least part of the tool (5) as captured by the respective 2D image of the intraoperative imaging data (ID) with the plurality of projections from the plurality of candidate perspectives.

8. The computer-implemented method of assisting positioning of a tool (5) with respect to a specific body part (202) of a patient (200) according to claim 1, wherein estimating the current position (5c) of the tool (5) is performed using a tool geometrical model indicative of a geometry of the tool (5), the step of estimating the current position (5c) of the tool (5) comprising at least one of the following steps:
a) comparing a projection of the tool geometrical model—onto the plane of one or more of the 2D images of the intraoperative imaging data (ID)—with the at least part of the tool (5) as captured by the respective 2D image of the intraoperative imaging data (ID); and
b) determining a position of the tool geometrical model that produces a projection onto the planes of the 2D images of the intraoperative imaging data (ID) that matches the at least part of the tool (5) as captured by the respective 2D image of the intraoperative imaging data (ID).

9. The computer-implemented method of assisting positioning of a tool (5) with respect to a specific body part (202) of a patient (200) according to claim 1, wherein generating positioning guidance data (GD) comprises overlaying the visual representation of the estimated current position (5c) of the tool (5) onto a visual representation of the reconstructed anatomical 3D shape (AS).

10. The computer-implemented method of assisting positioning of a tool (5) with respect to a specific body part (202) of a patient (200) according to claim 1, further comprising:
a) identifying, by the computing device (10), a prescribed position (5p) of the tool (5) with respect to the anatomical 3D shape (AS) of the specific body part (202); and
b) overlaying a visual representation of the prescribed position (5p) of the tool (5) onto a visual representation of the estimated current position (5c) of the tool (5).

11. The computer-implemented method of assisting positioning of a tool (5) with respect to a specific body part (202) of a patient (200) according to claim 10, wherein the step of identifying the prescribed position (5p) of the tool (5) comprises at least one of the following steps:
a) retrieving, by the computing device (10), the prescribed position (5p) of the tool (5) from a datastore comprised by or communicatively connected to the computing device (10); and
b) computing the prescribed position (5p) of the tool (5) by the computing device (10), the prescribed position (5p) of the tool (5) being determined by an optimization function based on the anatomical 3D shape (AS) of the body part as well as data indicative of a surgical procedure.

12. The computer-implemented method of assisting positioning of a tool (5) with respect to a specific body part (202) of a patient (200) according to claim 1, further comprising at least one of the following steps:
a) providing a tool (5) in accordance with a tool geometrical model;
b) capturing, using an imaging device (20), intraoperative imaging data (ID) capturing at least a body part of the patient (200) and at least a part of the tool (5); and
c) controlling, by the computing device (10), a display device (30) to display at least part of the guidance data (GD).

13. The computer-implemented method of assisting positioning of a tool (5) with respect to a specific body part (202) of a patient (200) according to claim 1, wherein the intraoperative imaging data (ID) comprises one or more of:
a) radiation-based images, in particular X-ray image(s) of the specific body part (202) of the patient (200) respectively a part of the tool (5);
b) ultrasound image(s) of the specific body part (202) of the patient (200) respectively a part of the tool (5);
c) arthroscopic image(s) of the specific body part (202) of the patient (200) respectively a part of the tool (5);
d) optical imagery of the specific body part (202) of the patient (200) respectively a part of the tool (5); and
e) cross-sectional imaging of the patient (200) respectively a part of the tool (5).

14. The computer-implemented method of assisting positioning of a tool (5) with respect to a specific body part (202) of a patient (200) according to claim 1, wherein the steps of: receiving intraoperative imaging data (ID); estimating a current position (5c) of the tool (5); and generating guidance data (GD) are carried out repeatedly or continuously for a period of time in preparation of/preceding a surgical treatment of the patient (200).

15. A computing device (10) comprising:
a) a data input interface (14) communicatively connectable with an imaging device (20) and configured to receive intraoperative imaging data (ID);
b) a data output interface (12) configured to transmit at least part of guidance data (GD) to a display device (30) communicatively connectable to the data output interface (12);
b) a processing unit (16); and
c) a storage unit (18) comprising instructions, which, when carried out by the processing unit (16), causes the computing device (10) to carry out the method according to claim 1.

16. A system (1) for assisting positioning of a tool (5) with respect to a specific body part (202) of a patient (200), the system (1) comprising:
a) a computing device (10) including a data input interface (14) communicatively connectable with an imaging device (20) and configured to receive intraoperative imaging data (ID); a data output interface (12) configured to transmit at least part of guidance data (GD) to a display device (30) communicatively connectable to the data output interface (12); a processing unit (16); and a storage unit (18) comprising instructions;
b) an imaging device (20) arranged and configured to capture intraoperative imaging data (ID) comprising 2D images, the intraoperative imaging data (ID) comprising 2D images, a plurality of the 2D images capturing the specific body part (202) of the patient (200) from a plurality of different perspectives with respect to the specific body part (202) of the patient (200) and one or more of the plurality of the 2D images capturing at least a part of the tool (5) from at least one perspective; and
c) a display device (30) configured to display a human-interpretable representation of at least part of the guidance data (GD), the display device (30) being communicatively connected to the data output interface (12) of the computing device (10),
the system (1) being configured to carry out the method according to claim 1 any one of the claims 1 to 14.

17. The system (1) for assisting positioning of a tool (5) with respect to a specific body part (202) of a patient (200) according to claim 16, the system (1) further comprising a tool (5) having a geometry corresponding to a tool geometrical model.

18. A computer program product, comprising instructions, which, when carried out by a processing unit (16) of a computing device (10), cause the computing device (10) to carry out the method according to claim 1.

19. The computer program product according to claim 18, wherein the instructions comprise an artificial-intelligence based algorithm corresponding to a specific body part (202) of a patient (200), the artificial-intelligence based algorithm having been trained using a multitude of annotated imaging data sets capturing body parts corresponding to the specific body part (202) of the patient (200), wherein the annotations comprise data identifying and/or describing properties of the body part.
